(19) United States
(12) Patent Application Publication
(10) Pub. No.: US 2015/0258226 A1
(43) Pub. Date: Sep. 17, 2015

(54) SUBSTRATE STORING CASE, SUBSTRATE CLEANING APPARATUS AND SUBSTRATE STORING CASE CLEANING APPARATUS

(71) Applicant: KABUSHIKI KAISHA TOSHIBA, Tokyo (JP)

(72) Inventors: Yukio Oppata, Chiba (JP); Hideaki Sakurai, Kawasaki (JP); Shingo Kanamitsu, Kawasaki (JP); Tomohiro Tsutsui, Setagaya (JP); Kazuki Hagihara, Kawasaki (JP)

(21) Appl. No.: 14/479,742

(22) Filed: Sep. 8, 2014

(30) Foreign Application Priority Data
Mar. 13, 2014 (JP) 2014-050857

(51) Int. Cl. A61L 2/04; B65D 81/30; A61L 2/08; B65D 25/10; B01D 46/00; B08B 7/00; B65D 13/02; B65D 81/18

(52) U.S. Cl. CPC A61L 2/04; B65D 13/02; B65D 81/30; B65D 81/18; B65D 25/10; B01D 46/00; B08B 7/005; A61L 2/085

(57) ABSTRACT

The substrate storing case includes a base being made of quartz glass, and having a supporting part that is formed on an upper surface thereof and supports a substrate. The substrate storing case includes a top cover being made of quartz glass, and being in contact with the base to cover the substrate on the upper surface of the base. The substrate includes a first absorptive member that absorbs infrared rays and generates heat. The base or the top cover has an intake port that is in communication with a space enclosed by the upper surface of the base and the top cover and is capable of being opened and closed, and an outlet port that is in communication with the space and is capable of being opened and closed.

### CROSS-REFERENCE TO RELATED APPLICATION

This application is based upon and claims the benefit of priority from the prior Japanese Patent Application No. 2014-050857, filed on Mar. 13, 2014, the entire contents of which are incorporated herein by reference.

### BACKGROUND

1. Field

Embodiments described herein relate generally to a substrate storing case, a substrate cleaning apparatus and a substrate storing case cleaning apparatus.

2. Background Art

With the recent advance of pattern micromachining, the manufacturing cost has become more dependent on the problem of defects of a substrate (lithography original plate).

Such defects of a substrate can be caused not only by particles adhering to the substrate but also impurities or nanoparticles in the environment to which the substrate is exposed.

Therefore, it is increasingly important to control impurities in the environment to which the substrate is exposed or to control particles having sizes on the order of nanometers adhering to the substrate.

For example, controlling the environment to which the substrate is exposed involves controlling the environment in a clean room, the environment in a processing device, and the environment in a substrate storing case used to transport the substrate.

The substrate storing case used to transport or store the substrate is typically made of a resin material (polycarbonate) because the resin material can be easily shaped, is inexpensive, is unlikely to produce outgas, is highly resistant to impact, and is highly resistant to cleaning, for example.

However, when the substrate storing case made of polycarbonate is cleaned, the substrate storing case cannot be heated to 100 degrees C. or higher because of the heat resistance of polycarbonate.

Therefore, there is a problem that it is difficult to remove, by heating, impurities or the like from the environment that adhere to the substrate storing case made of a resin.

### BRIEF DESCRIPTION OF THE DRAWINGS

FIG. 1 is a diagram showing an example of a configuration of the substrate storing case 100 according to a first embodiment;

FIG. 2 is a diagram showing an example of a configuration of the substrate cleaning apparatus 1000 with which the substrate storing case 100 shown in FIG. 1 is used;

FIG. 3 is a diagram showing an example of a configuration of a substrate storing case 200 according to the second embodiment;

FIG. 4 is a diagram showing an example of a configuration of a substrate storing case 300 according to the third embodiment;

FIG. 5 is a diagram showing an example of a configuration of a substrate storing case 400 according to the fourth embodiment;

FIG. 6 is a diagram showing an example of a configuration of a substrate storing case 500 according to the fifth embodiment; and

FIG. 7 is a diagram showing an example of a configuration of the substrate cleaning apparatus 2000 with which the substrate storing case 500 shown in FIG. 6 is used.

### DETAILED DESCRIPTION

A substrate storing case according to an embodiment includes a base being made of quartz glass, and the base having a supporting part that is formed on an upper surface thereof and supports a substrate. The substrate storing case includes a top cover being made of quartz glass, and being in contact with the base to cover the substrate on the upper surface of the base. The substrate includes a first absorptive member that absorbs infrared rays and generates heat. The base or the top cover has an intake port that is in communication with a space enclosed by the upper surface of the base and the top cover and is capable of being opened and closed, and an outlet port that is in communication with the space and is capable of being opened and closed.

In the following, embodiments will be described with reference to the drawings.

### First Embodiment

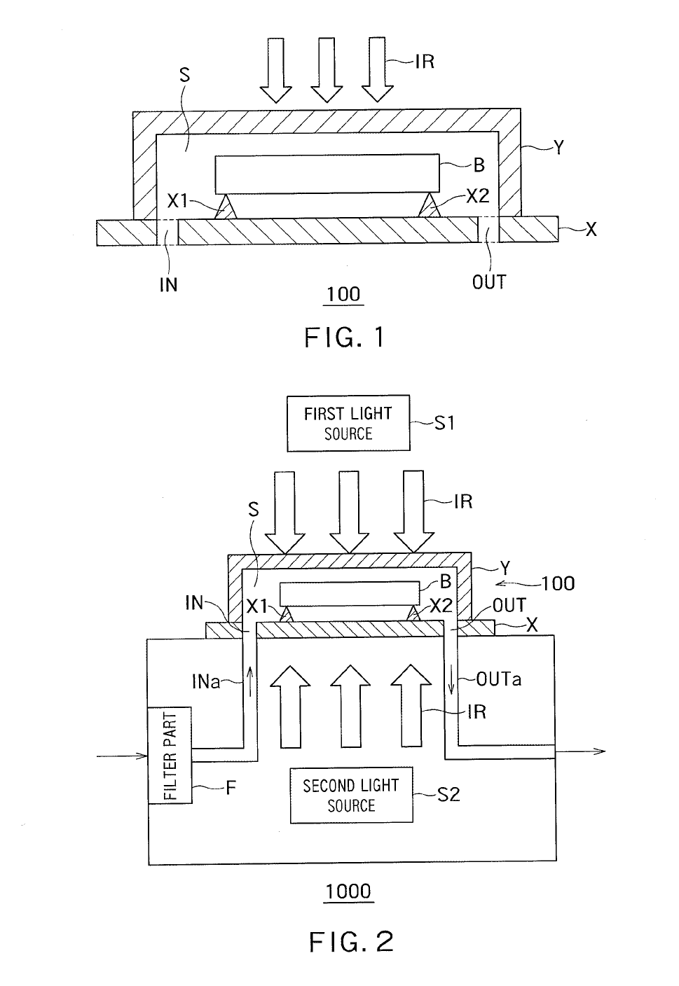

FIG. 1 is a diagram showing an example of a configuration of the substrate storing case 100 according to a first embodiment.

As shown in FIG. 1, the substrate storing case 100 that stores a substrate “B” includes a base “X” and a top cover “Y”.

The base “X” is made of quartz glass, which transmits infrared rays “IR”. The base “X” has supporting parts “X1” and “X2” that support the substrate “B” from below on the upper surface thereof.

The top cover “Y” is also made of quartz glass, which transmits infrared rays “IR”. The top cover “Y” is in contact with the base “X” when the top cover “Y” covers the substrate “B” on the base “X”.

The substrate “B” is held inside the substrate storing case 100 and thereby separated from the outside air.

As shown in FIG. 1, the base “X” has an intake port “IN” that is in communication with a space “S” enclosed by the base “X” and the top cover “Y” and is capable of being opened and closed and an outlet port “OUT” that is in communication with the space “S” and is capable of being opened and closed. However, the top cover “Y” may have an intake port “IN” that is in communication with the space “S” enclosed by the base “X” and the top cover “Y” and is capable of being opened and dosed and an outlet port “OUT” that is in communication with the space “S” and is capable of being opened and dosed.

The intake port “IN” is intended to introduce the outside air with impurities removed into the substrate storing case 100. The outlet port “OUT” is intended to discharge the atmosphere in the substrate storing case 100 to the outside.

When the substrate “B” is stored (during each processing step or when the substrate “B” is set in a processing device) or transported, the intake port “IN” and the outlet port “OUT” are closed.

In this way, the space “S” enclosed by the base “X” and the top cover “Y” of the substrate storing case 100 is hermetically closed.

On the other hand, as described later, when the substrate “B” or the substrate storing case 100 is cleaned, the intake port “IN” and the outlet port “OUT” are opened.

In this way, the outside air with impurities removed can be introduced into the substrate storing case 100, and the atmosphere in the substrate storing case 100 can be discharged to the outside.

The substrate “B” can be placed in the substrate storing case 100 when the base “X” and the top cover “Y” are at least partially separated from each other.

At least a part of the substrate “B” is formed by an absorptive member that absorbs infrared rays “IR” and generates heat. The substrate “B” is a lithography original plate, for example. A pattern formed on the substrate “B” (a circuit pattern on a lithography original plate, for example) is formed by the absorptive member. The absorptive member contains chromium. In the example shown in FIG. 1, the lithography original plate is a photomask.

As described above, the base “X” and the top cover “Y” forming the substrate storing case 100 are made of quartz glass. As a result, compared with a case where the substrate storing case is made of a common resin, production of impurities including organic substances can be suppressed.

Next, an example of a configuration of a substrate cleaning apparatus 1000 with which the substrate storing case 100 shown in FIG. 1 is used will be described.

FIG. 2 is a diagram showing an example of a configuration of the substrate cleaning apparatus 1000 with which the substrate storing case 100 shown in FIG. 1 is used.

As shown in FIG. 2, the substrate cleaning apparatus 1000 includes a filter part “F”, an introducing part “INa”, a sucking part “OUTa”, a first light source “S1”, and a second light source “S2”, for example.

The filter part “F” filters the outside air.

The introducing part “INa” introduces the outside air filtered by the filter part “F” into the intake port “IN” of the substrate storing case 100.

The sucking part “OUTa” sucks out the atmosphere in the space “S” in the substrate storing case 100 through the outlet port “OUT” of the substrate storing case 100.

The first light source “S1” emits infrared rays “IR” to the substrate storing case 100 from the side of the top cover “Y” so as to irradiate the upper surface of the substrate “B” housed in the substrate storing case 100 with the infrared rays “IR”.

The second light source “S2” emits infrared rays “IR” to the substrate storing case 100 from the side of the base “X” so as to irradiate the lower surface of the substrate “B” housed in the substrate storing case 100 with the infrared rays “IR”.

Next, an example of an operation of the substrate cleaning apparatus 1000 configured as described above cleaning the substrate “B” and the substrate storing case 100 by heating will be described.

As shown in FIG. 2, first, the substrate storing case 100 having the substrate “B” housed therein is placed in the substrate cleaning apparatus 1000.

The first light source “S1” then emits infrared rays “IR” to the substrate storing case 100 from the side of the top cover “Y” so as to irradiate the upper surface of the substrate “B” housed in the substrate storing case 100 with the infrared rays “IR”.

In addition, the second light source “S2” emits infrared rays “IR” to the substrate storing case 100 from the side of the base “X” so as to irradiate the lower surface of the substrate “B” housed in the substrate storing case 100 with the infrared rays “IR”.

The absorptive member of the substrate “B”, an absorptive member of the base “X” and an absorptive member of the top cover “Y” absorb the infrared rays “IR” and generate heat. That is, the substrate “B” and the substrate storing case 100 are heated by themselves.

Alternatively, an absorptive member that absorbs infrared rays “IR” may be applied or attached to the base “X” or the top cover “Y”. In that case, the substrate storing case 100 is heated when the substrate storing case 100 is externally irradiated with infrared rays “IR”.

In particular, the absorptive member is disposed at a part of the base “X” or the top cover “Y” where the absorptive member does not block the infrared rays “IR” externally applied to the substrate “B”. In this way, the substrate storing case 100 can be heated while heating the substrate “B” with the infrared rays “IR”.

In this way, impurities or nanoparticles can be separated from the surface of the interior of the substrate storing case 100 or the surface of the substrate “B”.

Meanwhile, the intake port “IN” is opened to establish communication between the introducing part “INa” and the space “S” in the substrate storing case 100, and the outlet port “OUT” is opened to establish communication between the sucking part “OUTa” and the space “S” in the substrate storing case 100.

Then, the introducing part “INa” introduces the outside air filtered by the filter part “F” into the intake port “IN” of the substrate storing case 100. In this way, the outside air with impurities filtered out is supplied to the space “S”.

In addition, the sucking part “OUTa” sucks out the atmosphere (air) in the space “S” in the substrate storing case 100 through the outlet port “OUT” of the substrate storing case 100. In this way, the atmosphere containing impurities is discharged from the space “S” to the outside.

In this way, the substrate cleaning apparatus 1000 discharges the impurities or nanoparticles removed by heating to the outside of the substrate storing case 100 through the sucking part “OUTa”.

In this way, impurities or nanoparticles on the surface of the substrate “B” or the inner surface of the substrate storing case 100 can be separated from the surface into the space “S” and removed from the space “S” in the substrate storing case 100, thereby reducing the probability of occurrence of defects of a pattern on the photomask or template or occurrence of adhesion of impurities or nanoparticles to the pattern of the photomask or template.

If the substrate “B” is not stored in the substrate storing case 100, the substrate cleaning apparatus 1000 configured as described above serves as a substrate storing case cleaning apparatus that cleans the substrate storing case 100.

Since the substrate storing case 100 is made of quartz glass as described above, the substrate storing case 100 can be heated to a higher temperature than the conventional substrate storing case made of a common resin. This ensures that impurities or nanoparticles can be efficiently separated off.

The maximum temperature at which quartz glass can be continuously used is approximately 900 degrees C. Therefore, the heating temperature of the substrate storing case 100 is controlled to fall within a range from room temperature (20 degrees C.) to 900 degrees C.

The impurities described above mainly include toluene, ethylbenzene, xylene, benzaldehyde, dichlorobenzene, ethylhexanol, benzoic acid, butanediol, trimethylbenzene, nonanol, butoxyethoxyethanol, tertiary butyl hydroxymethyl cyclohexadiene, trichlorfon, dibutyl phthalate, dioctyl phthalate, ammonia, amines, organic amines, organic acids, fluorine ions and compounds thereof, chlorine ions and compounds thereof, and sulfate ions and compounds thereof, for example.

As described above, the substrate can be heated without opening the substrate storing case. This can prevent adhesion or re-adhesion of contaminants, such as organic substances originating from the interior of the substrate storing case or the surface of the substrate, to the surface of the substrate, thereby reducing the probability of occurrence of defects of the substrate.

In addition, the atmosphere in the substrate storing case is discharged. This can prevent adhesion or re-adhesion of the contaminants to the surface of the substrate, thereby reducing the probability of occurrence of defects of the substrate (such as a pattern defect of a lithography original plate).

In short, the substrate storing case according to the first embodiment can reduce the probability of occurrence of defects of the substrate.

### Second Embodiment

In a second embodiment, an example of a configuration of a substrate storing case in the case where the substrate has a recess formed in the middle of the lower surface will be described.

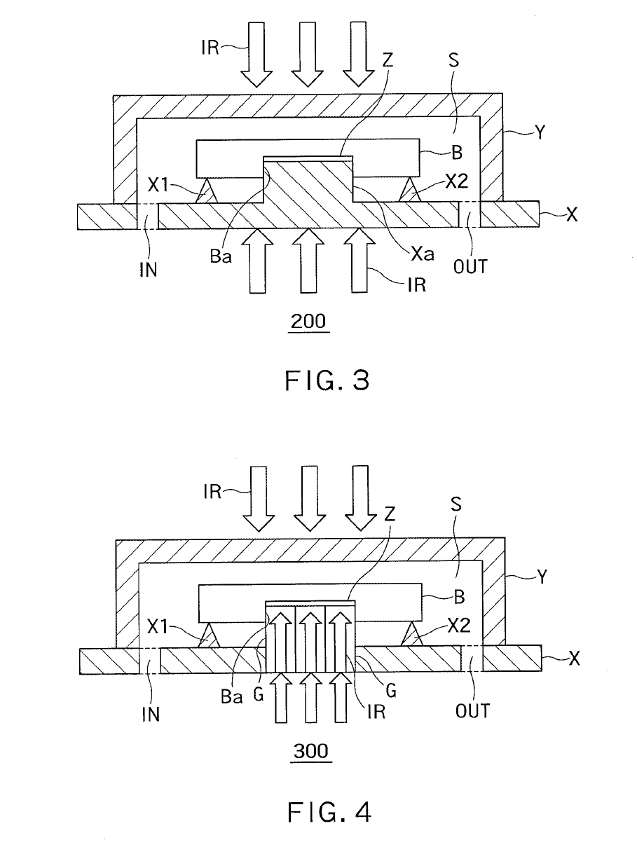

FIG. 3 is a diagram showing an example of a configuration of a substrate storing case 200 according to the second embodiment. In FIG. 3, the same reference symbols as those in FIG. 1 denote the same components as those in the first embodiment. The substrate storing case 200 shown in FIG. 3 is used with the substrate cleaning apparatus 1000 shown in FIG. 2, as with the substrate storing case 100 according to the first embodiment.

As shown in FIG. 3, the substrate “B” has a recess “Ba” formed in the middle of the lower surface thereof. In the example shown in FIG. 3, the substrate (lithography original plate) “B” is a template for nanoimprint.

The base “X” has a projection “Xa”, which is shaped to conform to the recess “Ba” of the substrate “B”, formed on the upper surface thereof.

The recess “Ba” of the substrate “B” may be externally irradiated with infrared rays “IR” through the projection “Xa” on the base “X”.

Then, the absorptive member of the substrate “B” absorbs the infrared rays “IR” and generates heat.

An auxiliary absorptive member “Z” that absorbs infrared rays “IR” and generates heat is disposed on the surface of the projection “Xa”. That is, the auxiliary absorptive member “Z” is disposed in contact with or close to the substrate “B”.

The auxiliary absorptive member Z″ may be externally irradiated with infrared rays “IR” through the projection “Xa” of the base “X”.

Then, the auxiliary absorptive member “Z” absorbs the infrared rays “IR” and generates heat.

The remainder of the configuration and functionality of the substrate storing case 200 is the same as that of the substrate storing case according to the first embodiment.

That is, as with the substrate storing case 100 according to the first embodiment, the substrate storing case 200 allows heating of the substrate without opening the substrate storing case. This can prevent adhesion or re-adhesion of contaminants, such as organic substances originating from the interior of the substrate storing case or the surface of the substrate, to the surface of the substrate, thereby reducing the probability of occurrence of defects of the substrate.

In addition, the atmosphere in the substrate storing case is discharged. This can prevent adhesion or re-adhesion of the contaminants to the surface of the substrate, thereby reducing the probability of occurrence of defects of the substrate (such as a pattern defect of a lithography original plate).

In short, the substrate storing case according to the second embodiment can reduce the probability of occurrence of defects of the substrate.

### Third Embodiment

In a third embodiment, an example of a configuration of another substrate storing case in the case where the substrate “B” has the recess “Ba” formed in the middle of the lower surface will be described.

FIG. 4 is a diagram showing an example of a configuration of a substrate storing case 300 according to the third embodiment. In FIG. 4, the same reference symbols as those in FIG. 3 denote the same components as those in the second embodiment. The substrate storing case 300 shown in FIG. 4 is used with the substrate cleaning apparatus 1000 shown in FIG. 2, as with the substrate storing case 200 according to the second embodiment.

As shown in FIG. 4, the substrate “B” has the recess “Ba” formed in the middle of the lower surface thereof.

The base “X” has a light guide “G”, which is shaped to conform to the recess “Ba” of the substrate “B”, formed in the middle thereof. That is, the substrate storing case 300 differs from the substrate storing case according to the first embodiment in that the substrate storing case 300 further has the light guide “G” that guides infrared rays “IR” from the outside of the substrate storing case 300 to the absorptive member or auxiliary absorptive member “Z” of the substrate “B”.

The recess “Ba” of the substrate “B” is externally irradiated with infrared rays “IR” through the light guide “G”.

Then, the absorptive member of the substrate “B” absorbs the infrared rays “IR” and generates heat.

The auxiliary absorptive member “Z” that absorbs infrared rays and generates heat may be disposed on the upper surface of the light guide “G”. That is, the auxiliary absorptive member “Z” is disposed in contact with or close to the substrate “B”.

The auxiliary absorptive member Z″ may be externally irradiated with infrared rays “IR” through the light guide “G” of the base “X”.

Then, the auxiliary absorptive member “Z” absorbs the infrared rays “IR” and generates heat.

The remainder of the configuration and functionality of the substrate storing case 300 is the same as that of the substrate storing case according to the second embodiment.

That is, the substrate storing case according to the third embodiment can reduce the probability of occurrence of defects of the substrate, as with the substrate storing case according to the second embodiment.

### Fourth Embodiment

In a fourth embodiment, an example of a configuration of another substrate storing case in the case where the substrate “B” has the recess “Ba” formed in the middle of the lower surface will be described.

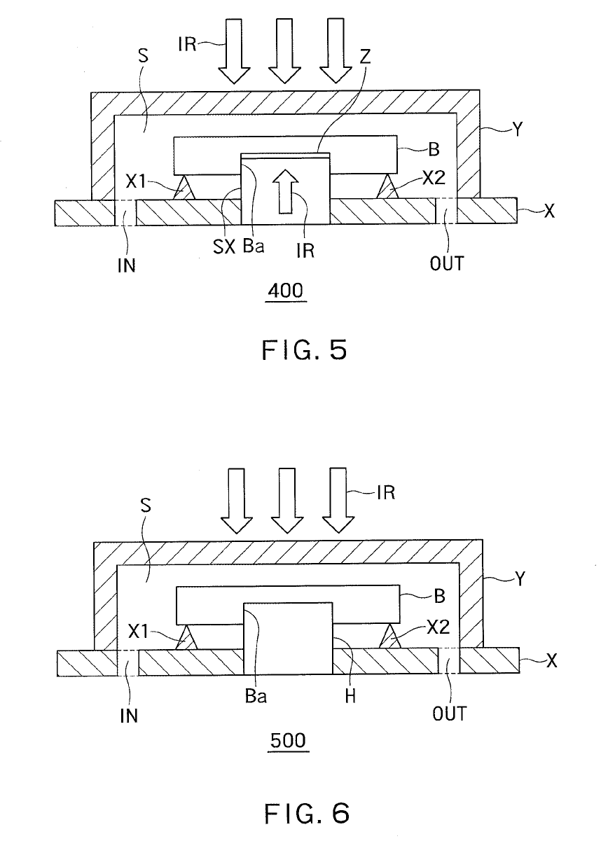

FIG. 5 is a diagram showing an example of a configuration of a substrate storing case 400 according to the fourth embodiment. In FIG. 5, the same reference symbols as those in FIG. 3 denote the same components as those in the second embodiment. The substrate storing case 400 shown in FIG. 5 is used with the substrate cleaning apparatus 1000 shown in FIG. 2, as with the substrate storing case 200 according to the second embodiment.

As shown in FIG. 5, the substrate “B” has the recess “Ba” formed in the middle of the lower surface thereof.

The base “X” has an internal light source “SX”, which is shaped to conform to the recess “Ba” of the substrate “B”, provided in the middle thereof. That is, the substrate storing case 400 differs from the substrate storing case according to the first embodiment in that the substrate storing case 400 further has the internal light source “SX” that emits infrared rays “IR” to the substrate “B”.

The internal light source “SX” irradiates the recess “Ba” of the substrate “B” with infrared rays “IR”. Then, the absorptive member of the substrate “B” absorbs the infrared rays “IR” and generates heat.

An auxiliary absorptive member “Z” that absorbs infrared rays “IR” and generates heat may be disposed on the upper surface of the internal light source “SX”. That is, the auxiliary absorptive member “Z” is disposed in contact with or close to the substrate “B”.

The internal light source “SX” may irradiate the auxiliary absorptive member Z″ with the infrared rays “IR”.

Then, the auxiliary absorptive member “Z” absorbs the infrared rays “IR” and generates heat.

The remainder of the configuration and functionality of the substrate storing case 400 is the same as that of the substrate storing case according to the second embodiment.

That is, the substrate storing case according to the fourth embodiment can reduce the probability of occurrence of defects of the substrate, as with the substrate storing case according to the second embodiment.

### Fifth Embodiment

In a fifth embodiment, an example of a configuration of another substrate storing case in the case where the substrate “B” has the recess “Ba” formed in the middle of the lower surface will be described.

FIG. 6 is a diagram showing an example of a configuration of a substrate storing case 500 according to the fifth embodiment. In FIG. 6, the same reference symbols as those in FIG. 3 denote the same components as those in the second embodiment.

As shown in FIG. 6, the substrate “B” has the recess “Ba” formed in the middle of the lower surface thereof.

The base “X” has a heating part “H”, which is shaped to conform to the recess “Ba” of the substrate “B”, provided in the middle thereof. That is, the substrate storing case 500 differs from the substrate storing case according to the first embodiment in that the substrate storing case 500 further has the heating part “H” that is disposed in contact with or close to the substrate “B” and heats the substrate “B”.

The remainder of the configuration and functionality of the substrate storing case 500 is the same as that of the substrate storing case according to the second embodiment.

Next, an example of a configuration of a substrate cleaning apparatus 2000 with which the substrate storing case 500 shown in FIG. 6 is used will be described.

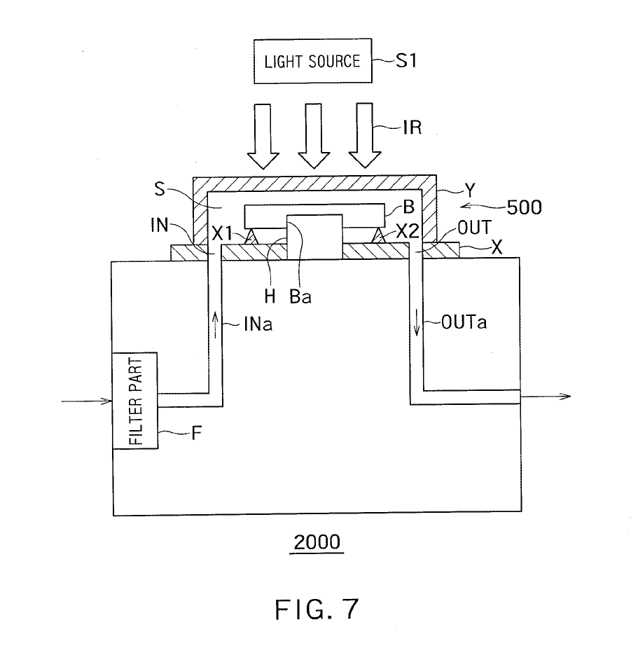

FIG. 7 is a diagram showing an example of a configuration of the substrate cleaning apparatus 2000 with which the substrate storing case 500 shown in FIG. 6 is used.

As shown in FIG. 7, the substrate cleaning apparatus 2000 includes the filter part “F”, the introducing part “INa”, the sucking part “OUTa” and the first light source “S1”, for example. That is, the substrate cleaning apparatus 2000 differs from the substrate cleaning apparatus 1000 according to the first embodiment in that the second light source “S2” is omitted.

The filter part “F” filters the outside air.

The introducing part “INa” introduces the outside air filtered by the filter part “F” into the intake port “IN” of the substrate storing case 500.

The sucking part “OUTa” sucks out the atmosphere in the space “S” in the substrate storing case 500 through the outlet port “OUT” of the substrate storing case 500.

The first light source “S1” emits infrared rays “IR” to the substrate storing case 500 from the side of the top cover “Y” so as to irradiate the upper surface of the substrate “B” housed in the substrate storing case 500 with the infrared rays “IR”.

The remainder of the configuration and functionality of the substrate cleaning apparatus 2000 is the same as that of the substrate cleaning apparatus 1000 according to the first embodiment.

Next, an example of an operation of the substrate cleaning apparatus 2000 configured as described above cleaning the substrate “B” and the substrate storing case 500 by heating will be described.

As shown in FIG. 7, first, the substrate storing case 500 having the substrate “B” housed therein is placed in the substrate cleaning apparatus 2000.

The first light source “S1” then emits infrared rays “IR” to the substrate storing case 500 from the side of the top cover “Y” so as to irradiate the upper surface of the substrate “B” housed in the substrate storing case 500 with the infrared rays “IR”.

The absorptive member of the substrate “B”, the absorptive member of the base “X” and the absorptive member of the top cover “Y” absorb the infrared rays “IR” and generate heat. That is, the substrate “B” and the substrate storing case 500 are heated by themselves.

In addition, the heating part “H” heats the substrate “B” housed in the substrate storing case 500.

In this way, impurities or nanoparticles can be separated from the surface of the interior of the substrate storing case 500 or the surface of the substrate “B”.

The remainder of the operation of the substrate cleaning apparatus 2000 is the same as that of the substrate cleaning apparatus 1000 according to the first embodiment.

If the substrate “B” is not stored in the substrate storing case 500, the substrate cleaning apparatus 2000 having the configuration and functionality described above serves as a substrate storing case cleaning apparatus that cleans the substrate storing case 500.

As described above, the substrate can be heated without opening the substrate storing case. This can prevent adhesion or re-adhesion of contaminants, such as organic substances originating from the interior of the substrate storing case or the surface of the substrate, to the surface of the substrate, thereby reducing the probability of occurrence of defects of the substrate.

In addition, the atmosphere in the substrate storing case is discharged. This can prevent adhesion or re-adhesion of the contaminants to the surface of the substrate, thereby reducing the probability of occurrence of defects of the substrate (such as a pattern defect of a lithography original plate).

In short, the substrate storing case according to the fifth embodiment can reduce the probability of occurrence of defects of the substrate.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A substrate storing case for storing a substrate, the substrate storing case comprising:
A base being made of quartz glass, the base having a supporting part that is formed on an upper surface thereof and supports the substrate,
A top cover being made of quartz glass, and being in contact with the base to cover the substrate on the upper surface of the base, and
wherein the substrate includes a first absorptive member that absorbs infrared rays and generates heat, and
the base or the top cover has an intake port that is in communication with a space enclosed by the upper surface of the base and the top cover and is capable of being opened and closed, and an outlet port that is in communication with the space and is capable of being opened and closed.

2. The substrate storing case according to claim 1, wherein the substrate is a lithography original plate.

3. The substrate storing case according to claim 2, wherein a pattern formed on the substrate is formed by the first absorptive member.

4. The substrate storing case according to claim 3, wherein the first absorptive member contains chromium.

5. The substrate storing case according to claim 1, further comprising:
a second absorptive member that absorbs infrared rays and generates heat that is disposed in contact with or close to the substrate.

6. The substrate storing case according to claim 5, wherein the substrate storing case further comprises an internal light source that irradiates the substrate with infrared rays.

7. The substrate storing case according to claim 6, wherein the internal light source irradiates the first absorptive member with infrared rays.

8. The substrate storing case according to claim 5, wherein the substrate storing case further comprises a light guide that guides infrared rays from outside the substrate storing case to the first absorptive member or the second absorptive member.

9. The substrate storing case according to claim 1, wherein the substrate storing case further comprises a heating part that heats the substrate that is disposed in contact with or close to the substrate.

10. A substrate cleaning apparatus, comprising:
a substrate storing case comprising a base and a top cover, the base being made of quartz glass, the base having a supporting part that is formed on an upper surface thereof and supports a substrate, the top cover being made of quartz glass, and being in contact with the base to cover the substrate on the upper surface of the base, the base or the top cover having an intake port that is in communication with a space enclosed by the upper surface of the base and the top cover and is capable of being opened and closed, and an outlet port that is in communication with the space and is capable of being opened and closed;
a filter part that filters an outside air;
an introducing part that introduces the outside air filtered by the filter part into the intake port of the substrate storing case;
a sucking part that sucks out an atmosphere in the space in the substrate storing case through the outlet port of the substrate storing case; and
a first light source that emits infrared rays to the substrate storing case from the side of the top cover so as to irradiate an upper surface of the substrate housed in the substrate storing case with the infrared rays,
wherein the substrate includes an first absorptive member that absorbs infrared rays and generates heat.

11. The substrate cleaning apparatus according to claim 10, further comprising:
a second light source that emits infrared rays to the substrate storing case from the side of the base so as to irradiate a lower surface of the substrate housed in the substrate storing case with the infrared rays.

12. The substrate cleaning apparatus according to claim 11, wherein the substrate is a lithography original plate.

13. The substrate cleaning apparatus according to claim 12, wherein a pattern formed on the substrate is formed by the first absorptive member.

14. The substrate cleaning apparatus according to claim 13, wherein the first absorptive member contains chromium.

15. The substrate cleaning apparatus according to claim 11, further comprising:
a second absorptive member that absorbs infrared rays and generates heat that is disposed in contact with or close to the substrate.

16. The substrate cleaning apparatus according to claim 15, wherein the substrate storing case further comprises an internal light source that irradiates the substrate with infrared rays.

17. The substrate cleaning apparatus according to claim 16, wherein the internal light source irradiates the first absorptive member with infrared rays.

18. The substrate cleaning apparatus according to claim 15, wherein the substrate storing case further comprises a light guide that guides infrared rays from outside the substrate storing case to the first absorptive member or the second absorptive member.

19. The substrate cleaning apparatus according to claim 11, wherein the substrate storing case further comprises a heating part that heats the substrate that is disposed in contact with or close to the substrate.

20. A substrate cleaning apparatus, comprising:
a substrate storing case comprising a base and a top cover, the base being made of quartz glass, the base having a supporting part that is formed on an upper surface thereof and supports a substrate, the top cover being made of quartz glass, and being in contact with the base to cover the substrate on the upper surface of the base, the base or the top cover having an intake port that is in communication with a space enclosed by the upper surface of the base and the top cover and is capable of being opened and closed, and an outlet port that is in communication with the space and is capable of being opened and closed;
a filter part that filters an outside air;
an introducing part that introduces the outside air filtered by the filter part into the intake port of the substrate storing case;
a sucking part that sucks out an atmosphere in the space in the substrate storing case through the outlet port of the substrate storing case; and
a first light source that emits infrared rays to the substrate storing case from the side of the top cover, the first light source being able to irradiate the substrate.
